(19) United States
(12) Patent Application Publication
(10) Pub. No.: US 2020/0085336 A1
(43) Pub. Date: Mar. 19, 2020

(54) DUAL-MODE EPIDERMAL CARDIOGRAM SENSOR

(71) Applicant: BOARD OF REGENTS, THE UNIVERSITY OF TEXAS SYSTEM, Austin, TX (US)

(72) Inventors: Nanshu LU, Austin, TX (US); Taewoo HA, Austin, TX (US); Pulin WANG, Austin, TX (US)

(21) Appl. No.: 16/616,538

(22) PCT Filed: May 22, 2018
(86) PCT No.: PCT/US2018/033861

§ 371 Date: Nov. 25, 2019

Related U.S. Application Data
(60) Provisional application No. 62/509,954, filed on May 23, 2017.

(51) Int. Cl. A61B 5/0408; A61B 5/00; A61B 5/11; A61B 5/021; A61B 5/0456; A61B 5/02; H01L 41/113; H01L 41/29; H01L 41/338

(52) U.S. Cl. CPC A61B 5/04085; A61B 5/6833; A61B 5/1102; A61B 5/02141; A61B 5/0456; H01L 41/193; H01L 41/1132; H01L 41/29; H01L 41/338; A61B 2562/0209; A61B 2562/125; A61B 5/02028

(57) ABSTRACT

A dual-mode epidermal sensor/electrode that, when worn on a human chest, is capable of synchronously/continuously monitoring electrical activity and mechano-acoustic activity of a cardiovascular system. The dual-mode epidermal sensor/electrode consists of a pair of stretchable electrocardiogram (ECG) electrodes made out of filamentary serpentine gold nano-membranes and a stretchable seismocardiogram (SCG) sensor comprising a filamentary serpentine PVDF. The dual-mode epidermal sensor/electrode is light, thin, flexible, and requires no operational power. The sensor can be laminated conformably and unobtrusively on a human chest to provide high fidelity ECG measurements and SCG measurements, and an estimated beat-to-beat blood pressure (BP). The dual-mode epidermal sensor is fabricated using a cost-effective, cut-and-paste method of construction.

### CROSS-REFERENCE TO RELATED APPLICATION

This application claims priority to and benefit of U.S. provisional patent application Ser. No. 62/509,954 filed May 23, 2017, which is fully incorporated by reference and made a part hereof.

### GOVERNMENT SUPPORT

This invention was made with government support under Grant No. N00014-16-1-2044 awarded by the Office of Naval Research and with government support under Grant No. FA9550-15-1-0112 awarded by the Air Force Office of Scientific Research. The government has certain rights in the invention.

### BACKGROUND

Cardiovascular diseases (CVD) are the leading cause of death in the United States and burden the nation hundreds of billions of dollars each year. To reduce the fatality and social costs caused by CVD, wearable continuous cardiovascular monitoring devices may be required for timely diagnosis and treatment of CVD.

Cardiovascular function may be monitored by sensing the electrical activity of the heart (e.g., electrocardiogram). In addition, cardiovascular function may be monitored by sensing mechanical or acoustic (i.e., mechano-acoustic) activity of the heart (e.g., phonocardiogram, seismocardiogram, and ballistocardiogram).

Sensing electrical and mechano-acoustic activity provides complementary information. For example, electrical activity may provide information regarding myocardial conduction, while mechanical activity may provide information regarding myocardial contraction.

Traditionally, various types of equipment have been used for measuring electrical activity and mechano-acoustic activity of a cardiovascular system. For example, an electrocardiogram (ECG) may be obtained using a wearable Holter monitor; a phonocardiogram (PCG) may be obtained using a stethoscope; a seismocardiogram (SCG) may be obtained using a digital accelerometer worn on the chest, and a ballistocardiogram (BCG) may be obtained using a swing bed or a force sensor placed on a weighing scale.

Measuring a blood pressure (BP) of a cardiovascular system has traditionally required a sphygmomanometer, which uses a pressurized cuff. The inflation/deflation of the cuff makes beat-to-beat BP measurements impossible. Beat-to-beat BP measurements, however, are highly desirable for quickly assessing various condition associated with CVDs (e.g., heart disease, stroke, end-stage renal failure, and peripheral vascular disease).

To sense a beat-to-beat BP, an ECG sensor (worn on the chest) and a photoplethysmogram (PPG) sensor (worn on a finger) may be used in combination to measure the time that takes for a pulse pressure (PP) waveform to propagate through a length of the arterial tree. This approach is not practical for long term sensing because of the inconvenient sensor configuration. In addition, conventional silver/silver chloride (Ag/AgCl) gel electrodes may result in skin irritation and dehydration may degrade their performance if worn for extended periods.

Recent research has been shown that synchronous measurements of (i) the electrical activity of the heart (i.e., ECG) and (ii) local vibrations of the chest wall induced by the seismic motion of the heart (i.e., SCG) or whole body movement caused by the ballistic forces on the heart (i.e., BCG) can be used to estimate a beat-to-beat BP.

Conventional approaches for synchronous measurements of ECG and SCG (or BCG) are still challenged by reliability, accuracy, cost, accessibility, and/or comfort. For example, mounting a rigid accelerometer or rigid piezoelectric transducer on a human chest to measure SCG is uncomfortable and not practical for extended periods.

A need therefore exists for an integrated, wearable SCG sensor and ECG electrodes patch for simultaneously measuring electrical and mechanical cardiovascular signals to estimate a beat-to-beat BP.

### SUMMARY

Accordingly, in one aspect, the present disclosure a dual-mode epidermal sensor for simultaneously measuring signals for electrocardiography (ECG) and seismocardiography (SCG). The dual-mode epidermal sensor includes a flexible substrate that adheres and conforms to an epidermis. An ECG sensor is formed from a pair of electrodes on the surface of the flexible substrate. Each electrode in the pair is configured in an electrode pattern that allows the ECG sensor to flex with the flexible substrate to conform to the epidermis. In addition, a SCG sensor is formed from a film of piezoelectric material on the surface of the flexible substrate. The piezoelectric material is configured in a piezo pattern that allows the SCG sensor to flex with the flexible substrate to conform to the epidermis.

In an exemplary embodiment, the flexible substrate of the dual-mode epidermal sensor is a hydrocolloid medical dressing with adhesive on one side to adhere to the epidermis. In a possible embodiment, the thickness of the hydrocolloid medical dressing is less than 50 microns with surface dimensions of approximately 65 millimeters by 40 millimeters.

In another exemplary embodiment, each electrode of the dual-mode epidermal sensor is a gold nano-membrane (NM) on a supporting layer of polyethylene terephthalate (PET). In some cases, the gold NM is approximately 100 nanometers (nm) thick.

In another exemplary embodiment of the dual-mode epidermal sensor, the electrode pattern on the surface of the flexible substrate is serpentine in shape and may include terminal pads for connection to an interconnect.

In another exemplary embodiment of the dual-mode epidermal sensor, the film of piezoelectric material is polyvinylidene fluoride (PVDF). In some cases, the PVDF is less than 30 microns thick.

In another exemplary embodiment of the dual-mode epidermal sensor, the piezo pattern on the surface of the flexible substrate is serpentine shaped and may include nickel copper electrodes on a top surface and a bottom surface of film of PVDF material.

In another exemplary embodiment of the dual-mode epidermal sensor, the SCG sensor is disposed between the ECG sensor's pair of electrodes on the surface of the flexible substrate because the pair of electrodes may be spaced apart by approximately 3 cm. In some cases, the SCG may be covered by a second flexible substrate to isolate it from the epidermis.

In another exemplary embodiment, the total thickness of the dual-mode epidermal sensor is less than 125 microns, and in some cases, the total mass of the dual-mode epidermal sensor is 150 milligrams or less.

In another exemplary embodiment, the electrode and the piezo pattern are serpentine patterns that have a radius of curvature of approximately 2 millimeters and a width to radius ratio of between 0.4 and 0.8.

In another aspect, the present disclosure embraces a method for fabricating a dual-mode epidermal sensor. The method includes creating a sheet of electrode by depositing gold onto a PET film. The sheet of electrode is then attached to a first dummy substrate and a pair of electrodes having a serpentine pattern that conforms to an epidermis to sense electrical signals are cut. The pair of electrodes are then transferred from the first dummy substrate to a flexible substrate. The method then includes attaching a film of PVDF to a second dummy substrate and a piezoelectric sensor that has a second serpentine pattern that conforms to an epidermis to sense mechanical perturbations is cut. The piezoelectric sensor is then transferred from the second dummy substrate to the flexible substrate between the pair of electrodes. Finally, the piezoelectric sensor on the flexible substrate is covered with a second flexible substrate.

In another aspect, the present disclosure embraces a method for using a dual-mode epidermal sensor. The method includes attaching a dual-mode epidermal sensor, having an electrocardiography (ECG) sensor and seismocardiography (SCG) sensor, to a chest, proximate to the heart. Then ECG test equipment is attached to the ECG sensor and SCG test equipment is connected to the SCG sensor to simultaneously measure an electrocardiogram and a seismocardiogram, respectively.

In an exemplary embodiment of the method for using the dual-mode epidermal sensor, the method also includes the step of computing a beat-to-beat blood pressure (BP) from the electrocardiogram and seismocardiogram.

The foregoing illustrative summary, as well as other exemplary objectives and/or advantages of the disclosure, and the manner in which the same are accomplished, are further explained within the following detailed description and its accompanying drawings.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

Figure (FIG.) 1 graphically depicts integrated sensor/electrodes for electro- and mechano-acoustic cardiovascular (EMAC) sensing according to an exemplary embodiment of the present disclosure.

FIG. 2 graphically depicts operations of a fabrication process for integrated sensor/electrodes for EMAC sensing according to an exemplary embodiment of the present disclosure.

FIG. 3 graphically depicts an exemplary integrated sensor/electrodes attached to a chest for simultaneously sensing ECG and SCG according to an exemplary implementation of the present disclosure.

FIG. 4 graphically depicts simultaneously measured ECG and SCG signals from integrated sensor/electrodes for EMAC sensing according to an exemplary implantation of the present disclosure.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬ from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

The present disclosure embraces an ultrathin (e.g., ˜122 um), stretchable (e.g., ˜60%) epidermal patch having integrated electrocardiogram (ECG) electrodes and a seismocardiogram (SCG) sensor for cardiovascular monitoring.

The SCG sensor and the ECG electrodes (i.e., ECG sensor) are integrated together on a single, wearable patch that, in an exemplary embodiment, measures 63.5 millimeters (mm)×38.1 mm×0.122 mm, though any size is contemplated within the scope of this disclosure. When applied to the chest, the patch may be used with test equipment to measure ECG and SCG synchronously. Accordingly, the patch may be referred to as a dual-mode (i.e., ECG and SCG) epidermal sensor.

The SCG sensor is a piezoelectric material (e.g., polyvinylidene fluoride) that converts mechanical energy to electrical energy and requires no power supply. The ECG electrodes are spatially separated gold film patterns that, when in contact with the skin, convey electrical signals from the body to a piece of test equipment.

The integrated sensor/electrodes for electro- and mechano-acoustic cardiovascular (EMAC) sensing offer several advantages over other devices/system for cardiovascular monitoring. First, the sensor/electrodes for EMAC sensing do not contain any rigid components. Second, the sensor/electrodes do not require any power for operation. Third, the sensor/electrodes can be laminated conformably and unobtrusively on a human chest, without a significant acoustic impedance mismatch resulting from the skin. Fourth, the sensor/electrodes can be used to synchronously measure ECG and SCG, which facilitates a means for estimating blood pressure. Fifth, the sensor and electrodes can be fabricated using a quick and cost-effective cut-and-paste process.

The sensor/electrodes are shaped in a serpentine pattern to provide flexibility, thereby replacing bulky and rigid alternatives (e.g., commercial accelerometers) that are typically used. As will be described further below, the serpentine pattern is selected to balance flexibility (i.e., comfort) with sensitivity (i.e., performance). In an exemplary embodiment, the sensor/electrodes have a mass of 150 milligrams (mg), a thickness of 122 microns (μm), and an effective modulus of 8.5 megapascals (MPa). These values represent the lightest and thinnest mechano-acoustic-electrophysiological (MAE) sensing platform known. The wearability and measurement versatility make the integrated sensor/electrodes suitable for most medical, health and/or fitness applications requiring cardiac monitoring.

As mentioned, the integrated sensor/electrodes can be used for synchronous ECG and SCG measurements. This aspect allows for the estimation of blood pressure (BP). To estimate BP, an ECG signal and an SCG signal are measured using the sensor/electrodes that are applied to the chest of a test subject. A time interval between the R peak of the measured ECG and the AC peak of the measured SCG represent the sum of the isovolumetric contraction time (IVCT) and the left ventricular ejection time (LVET). This time interval between the R peak and the AC peak is known as an “RAC.” It has been shown that RAC is highly correlated with systolic/diastolic blood pressure (BP). Accordingly, estimates of BP may be obtained using the RAC.

Beat-to-beat BP monitors have traditionally utilized a pulse transit time (PTT) to estimate BP. Measuring PTT, however, requires two sensors placed in different locations on the test subject. Accordingly, the measurement setup may require cumbersome wires or transceivers. Conversely, measuring beat-to-beat BP using an RAC derived from SCG/ECG signals obtained from an integrated patch requires a much simpler setup. This simpler setup is more comfortable to a test subject.

As mentioned, the integrated sensor/electrodes can be fabricated using a cut-and-paste method manufacturing process. This process is time effective and inexpensive because it can produce an integrated sensor/electrodes patch in an ambient environment in less than 20 minutes. This is an improvement over traditional microfabrication methods, such as photolithography, which require expensive materials, expensive tools, and long fabrication periods.

FIG. 1 graphically depicts integrated sensor/electrodes for electro- and mechano-acoustic cardiovascular (EMAC) sensing according to an exemplary embodiment of the present disclosure. The stretchable EMAC sensing patch (i.e., tattoo) 100 incorporates filamentary serpentine ribbons of 102, 104 approximately 100 nm thick gold (Au) nano-membranes (NMs) on approximately 12.5 μm thick supporting polyethylene terephthalate (PET), and approximately 28 μm thick filamentary-serpentine-shaped polyvinylidene fluoride (PVDF) with approximately 200 nm thick nickel-copper (Ni—Cu) electrodes 106, 108 on both top and bottom surfaces. The Au NMs 102, 104 and the PVDF is disposed on a layer of 47 μm thick soft, medical dressing (e.g., TAGADERM™). The Au NMs are exposed on one side to make direct contact with skin; however to avoid discharging through the skin, the PVDF has an additional covering layer of approximately 47 μm thick soft, medical dressing. Two Au electrodes (shown with arrows on sides) 102 are sufficient for ECG sensing because of the built-in noise removal capability of an instrumentation amplifier and post-denoising process. The two Au NM electrodes 102 are typically spaced apart by approximately 3 cm for ECG measurements. In the embodiment shown in FIG. 1, the SCG sensor 110 comprises the serpentine electrodes 104 in the center of the patch 100.

The total thickness of the integrated sensor/electrodes, including the double layer of TAGADERM™, is approximately 122 μm and the total mass is approximately 150 mg. Thus, laminating the sensor/electrodes on human skin imposes negligible mechanical constraints from arbitrary skin deformations. Even after severe skin deformation, the sensor/electrodes can remain fully conformed to the skin without delamination, slippage, or mechanical failure, which ensures high fidelity sensing.

To manufacture the integrated EMAC sensing tattoo, a dry, freeform cut-and-paste method can be used. Instead of using thermal release tape, weakly adhesive transfer tape (for example, TRANSFERRITE ULTRA™ 582U) can be used as the temporary support to avoid the thermal deformation of PVDF. The entire manufacturing process can be chemical-free, mask-free, and stencil-free and can be completed within 20 minutes.

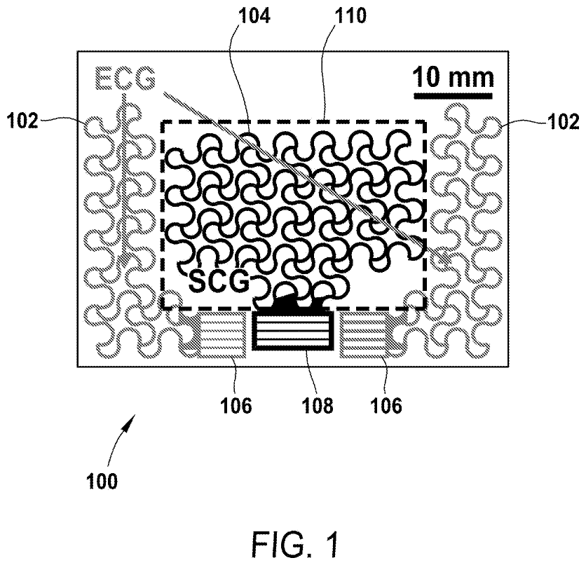

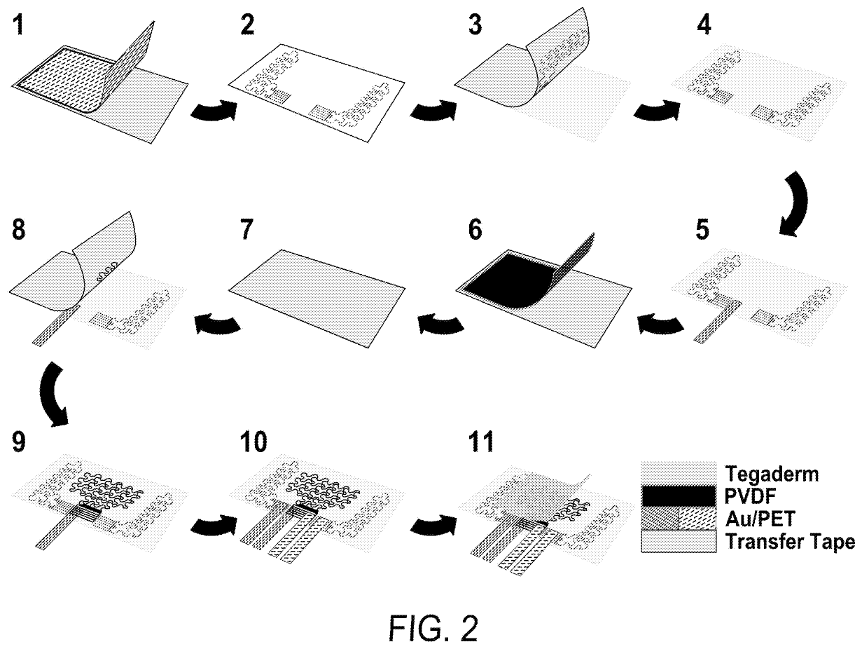

FIG. 2 graphically depicts operations of an embodiment of a fabrication process for integrated sensor/electrodes for EMAC sensing according to an exemplary embodiment of the present disclosure. The process requires four primary steps to create/transfer the ECG electrodes (e.g., Au NM) to a target substrate (e.g., TAGADERM™) and four primary steps to create/transfer the SCG sensor (e.g., PVDF) to the target substrate. The four steps are: (i) laminating a film (e.g., Au NM or PVDF) to a dummy substrate (see steps 1 and 6); (ii) cutting the film using a cutting machine (see steps 2 and 7); (iii) removing excess material after cutting; and (iv) transferring a remaining pattern to a target substrate (see steps 3 and 8).

In an exemplary implementation of the process, 100 nm Au is thermally deposited on a 12.5 μm PET film for support. To secure the film from misalignment during the cutting process, the 76.2 mm×50.8 mm Au/PET film is attached to a dummy substrate, which comprises 100 μm transfer tape (e.g., TRANSFERRITE-ULTRA™ 582U) and a 110 μm back-supporting film (e.g., INKPRESS MEDIA™). Within several minutes, the film can be carved using a cutting machine (e.g., SILHOUETTE-CAMEO™) with the cutting pattern prepared by a mechanical drafting program (e.g., AUTOCAD™). The blade depth setting on the cutter is established using software (e.g., SILHOUETTE STUDIO™) so as not to cut through the dummy substrate. After cutting, the pattern on the dummy substrate is transferred to the target substrate (e.g., TEGADERM™ 3M™) using the difference of adhesion force between the transfer tape (2.2 N/25 mm, peel adhesion @ 90°) and the target substrate (35.6 N, mean removal force).

After the Au/PET (i.e., electrode) pattern for ECG was transferred to the target substrate, four 25.4 mm×3.81 mm bridge electrodes are attached to connect the bottom electrode of PVDF film. The bridge electrodes are also made out of Au/PET and are strengthened using an attached 60 μm backing layer (e.g., AVERY™). The backing layer protects the Au/PET films from cracking when the flat flexible connector (FFC, Clincher Flex Connectors, AMPHENOL-FCI™) clutches the bridge electrode.

Similar to the Au/PET film (i.e., electrode) preparation, a 28.4 μm PVDF film (piezo film sheets, TE-CONNECTIVITY™) is attached to a dummy substrate and cut into a pattern by the cutting machine as described above. Next, the PVDF pattern is transferred to the target substrate that is aligned with the bridge electrode. Lastly, a covering layer (e.g., TEGADERM™ 3M™) is placed onto the patterned PVDF film to prevent the piezoelectric material from directly contacting the skin. The overall dimensions of the final sensor/electrodes are about 63.5 mm×38.1 mm×0.122 mm.

The patterns shown for the ECG electrodes and the SCG sensor in FIG. 1 are serpentine patterns. Compared with linear patterns, serpentine patterns can better stretch and flex with the skin; however serpentine patterns do not provide as high of a voltage output. One exemplary embodiment that provides a reasonable balance is a serpentine pattern with a width to radius of curvature (i.e., w/R) of 0.4 for a w=500 μm). The effective modulus of this pattern is 8.5 MPa, which is comparable to that of the stratum corneum of human skin.

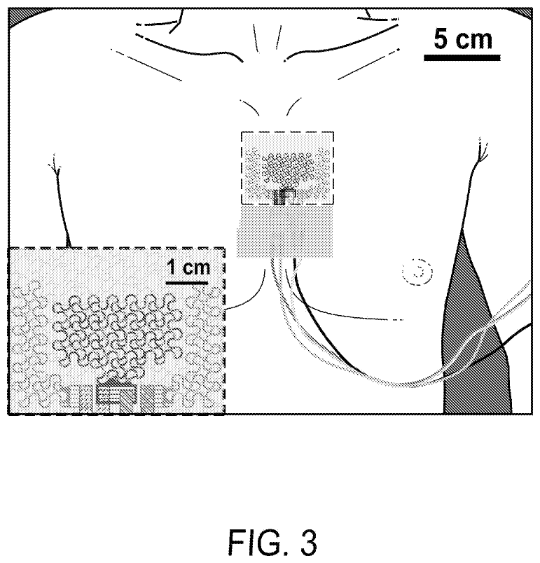

FIG. 3 graphically depicts an exemplary integrated sensor/electrodes attached to a chest for simultaneously sensing ECG and SCG according to an exemplary implementation of the present disclosure. Placement of the sensor/electrodes may be optimized to provide the strongest signals. FIG. 3 also illustrates interconnects (e.g., wires) attached to the bridge electrodes. The interconnects are also attached to DAQ test equipment (not shown).

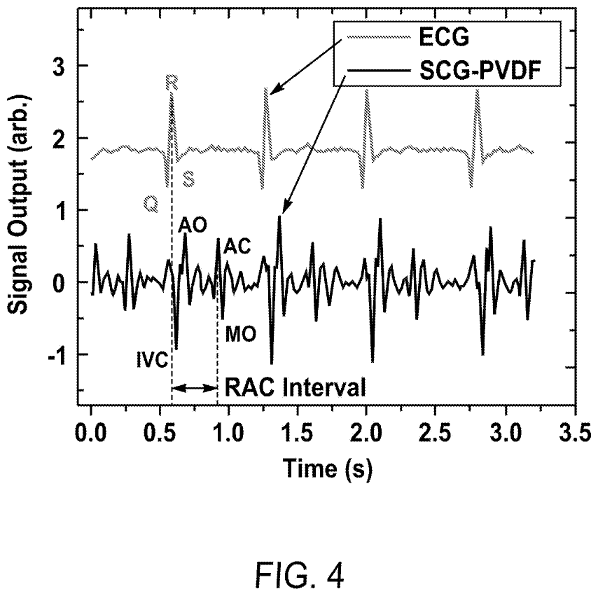

FIG. 4 graphically depicts simultaneously measured ECG and SCG signals from integrated sensor/electrodes for EMAC sensing according to an exemplary implantation of the present disclosure. The graphs illustrate synchronously measured ECG (top) and SCG (bottom) signals from the sensor/electrodes after signal processing. Q, R, and S peaks of ECG and AO (Aotic Valve Opening), IVC (Isovolumic Contraction), AC (Aortic Valve Closing) and MO (Mitral Valve Opening) peaks of SCG are labeled. Among the labeled features, the R peak of ECG and the AC peak of SCG are used for estimating BP. The R peak of ECG is the signature of the closure of the mitral valve and the 2nd peak of the PCG reflects the closure of the aortic valve, which is identical to the AC peak of the SCG. Hence, the time interval between the R peak of ECG and the AC peak of SCG is the RAC interval (i.e., systole) and consists of the isovolumic contraction time (IVCT) and the left ventricular ejection time (LVET). IVCT is the time from the mitral valve closure to the aortic valve opening and LVET is the time between the aortic valve opening and closing.

In the specification and/or figures, typical embodiments have been disclosed. The present disclosure is not limited to such exemplary embodiments. Those skilled in the art will also appreciate that various adaptations and modifications of the preferred and alternative embodiments described above can be configured without departing from the scope and spirit of the disclosure.

The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A dual-mode epidermal sensor for simultaneously measuring signals for electrocardiography (ECG) and seismocardiography (SCG), comprising
a flexible substrate that adheres and conforms to an epidermis;
an ECG sensor comprising a pair of electrodes, wherein each electrode forms an electrode pattern on a surface of the flexible substrate and flexes with the flexible substrate to conform to the epidermis; and
a SCG sensor comprising a film of piezoelectric material, wherein the film of piezoelectric material forms a piezo pattern on the surface of the flexible substrate and flexes with the flexible substrate to conform to the epidermis.

2. The dual-mode epidermal sensor according to claim 1, wherein: the flexible substrate is a hydrocolloid medical dressing with adhesive on one side to adhere to the epidermis.

3. The dual-mode epidermal sensor according to claim 1, wherein the flexible substrate has a thickness of less than 50 microns.

4. The dual-mode epidermal sensor according to claim 1, wherein the surface of the flexible substrate has dimensions of approximately 65 millimeters by 40 millimeters.

5. The dual-mode epidermal sensor according to claim 1, wherein each electrode in the pair of electrodes is a gold nano-membrane (NM) on a supporting layer of polyethylene terephthalate (PET).

6. The dual-mode epidermal sensor according to claim 5, wherein the gold nano-membrane is approximately 100 nanometers thick.

7. The dual-mode epidermal sensor according to claim 6, wherein the electrode pattern on the surface of the flexible substrate is serpentine shaped.

8. The dual-mode epidermal sensor according to claim 7, wherein each electrode pattern includes terminal pads for connection to an interconnect.

9. The dual-mode epidermal sensor according to claim 1, wherein the film of piezoelectric material is polyvinylidene fluoride (PVDF).

10. The dual-mode epidermal sensor according to claim 9, wherein the film of PVDF is less than 30 microns thick.

11. The dual-mode epidermal sensor according to claim 10, wherein the piezo pattern on the surface of the flexible substrate is serpentine shaped.

12. The dual-mode epidermal sensor according to claim 10, wherein the film of PVDF material comprises nickel copper (NiCu) electrodes on a top surface and a bottom surface of the film of PVDF material.

13. The dual-mode epidermal sensor according to claim 1, wherein the SCG sensor is disposed between the ECG sensor's pair of electrodes on the surface of the flexible substrate.

14. The dual-mode epidermal sensor according to claim 1, wherein a second flexible substrate covers the SCG sensor to isolate it from the epidermis.

15. The dual-mode epidermal sensor according to claim 14, wherein the total thickness of the dual-mode epidermal sensor is less than 125 microns.

16. The dual-mode epidermal sensor according to claim 14, wherein the total mass of the dual-mode epidermal sensor is 150 milligrams (mg) or less.

17. The dual-mode epidermal sensor according to claim 1, wherein the pair of electrodes are spaced apart by approximately 3 centimeters (cm) on the surface of the flexible substrate.

18. The dual-mode epidermal sensor according to claim 1, wherein the electrode pattern and the piezo pattern are serpentine patterns, wherein each serpentine has a radius of curvature of approximately 2 millimeters and wherein the serpentine has a width to radius ratio of between 0.4 and 0.8.

19. A method of fabricating a dual-mode epidermal sensor, the method comprising:
creating a sheet of electrode by depositing gold on a polyethylene terephthalate (PET) film;
attaching the sheet of electrode to a first dummy substrate;
cutting a pair of electrodes from the sheet of electrode wherein each of the pair of electrodes has a first serpentine pattern that conforms to an epidermis to sense electrical signals;
transferring the pair of electrodes from the first dummy substrate to a flexible substrate;
attaching a film of polyvinylidene fluoride (PVDF) to a second dummy substrate;
cutting a piezoelectric sensor from the PVDF film, wherein the piezoelectric sensor has a second serpentine pattern that conforms to an epidermis to sense mechanical perturbations;
transferring the piezoelectric sensor from the second dummy substrate to the flexible substrate between the pair of electrodes; and
covering the piezoelectric sensor on the flexible substrate with a second flexible substrate.

20. A method for using a dual-mode epidermal sensor, the method comprising:
attaching a dual-mode epidermal sensor to a chest, proximate to the heart, wherein the dual-mode epidermal sensor has a electrocardiography (ECG) sensor and seismocardiography (SCG) sensor;
connecting ECG test equipment to ECG sensor and SCG test equipment to the SCG sensor; and
simultaneously measuring an electrocardiogram and seismocardiogram using the ECG test equipment and the SCG test equipment, respectively.

21. The method for using a dual-mode epidermal sensor according to claim 20, further comprising:
computing a beat-to-beat blood pressure (BP) from the electrocardiogram and seismocardiogram.
